(19) United States
(12) Plant Patent Application Publication
(10) Pub. No.: US 2014/0096295 P1
(43) Pub. Date: Apr. 3, 2014

(54) Strawberry plant named 'RED MERLIN'

(50) ;

(71) Applicant: REDEVA LTD., Surbiton (GB)

(72) Inventors: Eva Izsak, Rehovot (IL); Ronald John McNicol, Dundee (GB)

(73) Assignee: Redeva Ltd., Surbiton (GB)

(21) Appl. No.: 13/573,619

(22) Filed: Sep. 28, 2012

(51) Int. Cl. A01H 5/00

(52) U.S. Cl. PLT/208

(57) ABSTRACT

This invention relates to a new and distinct variety of strawberry plant named ‘RED MERLIN’. The variety is characterized by its red fruit color, conical fruit shape, medium fruit size, early time of ripening, not remontant bearing, and flat globose plant habit.

### LATIN NAME OF THE GENUS AND SPECIES OF THE PLANT CLAIMED

Fragaria L.

### VARIETY DENOMINATION

‘RED MERLIN’

### BACKGROUND OF THE INVENTION

The present invention relates to a new and distinct strawberry variety named ‘RED MERLIN’. This new variety is a result of a controlled cross made between the strawberry variety designated ‘Sweet Charlie’ and the strawberry variety designated ‘Dorit’. The variety is botanically known as Fragaria L.

The new variety was asexually propagated. This propagation has demonstrated that the combination of traits disclosed herein as characterizing the new variety are fixed and remain true to type through successive generations of asexual reproduction.

### BRIEF SUMMARY OF THE INVENTION

The following traits have been repeatedly observed and are determined to be unique characteristics of ‘RED MERLIN’, which in combination distinguish this strawberry plant as a new and distinct variety:

1. Red fruit color and conical fruit shape;

2. Medium fruit size;

3. Early time of ripening;

4. Not remontant bearing; and

5. Flat globose plant habit.

### BRIEF DESCRIPTIONS OF THE PHOTOGRAPHS

The accompanying color photographs illustrate the overall appearance of typical specimens of the new strawberry variety ‘RED MERLIN’, as true as it is reasonably possible with color reproductions of this type. Color in the photographs may differ slightly from the color value cited in the botanical descriptions which accurately describe the color of ‘RED MERLIN’.

FIG. 1 shows a close up view of the typical fruit and flowers of ‘RED MERLIN’.

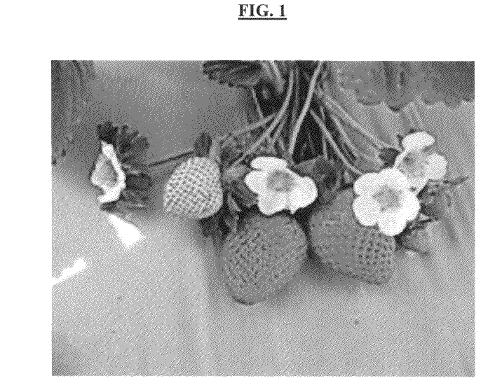

### DETAILED BOTANICAL DESCRIPTION

The new variety ‘RED MERLIN’ has not been observed under all possible environmental conditions. The characteristics of the new variety ‘RED MERLIN’ may vary in detail, depending upon variations in environmental factors, including weather (temperature, humidity and light intensity), day length, soil type and location. In addition, the characteristics of any parental variety or comparison variety included in the description of the present invention may vary in detail, depending upon variations in environmental factors, including weather (temperature, humidity and light intensity), day length, soil type and location.

Color terminology where noted follows The Royal Horticultural Society Colour Chart, London (2007).

The following table describes characteristics of the new strawberry ‘RED MERLIN’.

TABLE 1
FRUIT CHARACTERISTICS

| Characteristic | ‘RED MERLIN’ |
|---|---|
| Color of mature fruit | Red |
| Fruit size | Medium |
| Predominant fruit shape | Conical |
| Time of Ripening | Early |
| Type of bearing | Not remontant |

TABLE 2
PLANT CHARACTERISTICS

| Characteristic | ‘RED MERLIN’ |
|---|---|
| Plant habit | Flat Globose |

TABLE 3
FOLIAGE CHARACTERISTICS

| Characteristic | ‘RED MERLIN’ |
|---|---|
| Petiole: | |
| Attitude of hairs | Slightly outward |

TABLE 4
FLOWER CHARACTERISTICS

| Characteristic | ‘RED MERLIN’ |
|---|---|
| Position of inflorescence | Above |
| relative to foliage | |

We claim:

1. A new and distinct strawberry plant named ‘RED MERLIN’, as herein described and illustrated by the characteristics set forth above.
